(19) United States
(12) Patent Application Publication
(10) Pub. No.: US 2021/0302423 A1
(43) Pub. Date: Sep. 30, 2021

(54) Pathogen Mitigating Wearable Device

(71) Applicant: Robert Joe Alderman, Poteet, TX (US)

(72) Inventor: Robert Joe Alderman, Poteet, TX (US)

(21) Appl. No.: 17/212,427

(22) Filed: Mar. 25, 2021

Related U.S. Application Data
(60) Provisional application No. 63/100,687, filed on Mar. 26, 2020.

(51) Int. Cl. G01N 33/569; G01N 33/543

(52) U.S. Cl. CPC G01N 33/569; G01N 33/54386

(57) ABSTRACT

A device for mitigating the replication of pathogens at a user's nose. The device may include architecture and techniques for both introducing heat to nostril locations while at the same time, mitigating the temperature level at the nose for sake of long-term user comfort. In one embodiment this may be achieved through a device that utilizes a heat-generating substance layer in combination with a temperature modulating substance layer that employs a phase change material to attain the temperature modulation. In this type of construction, the phase change material may also be employed to take on and store heat that is later released to extend the effective life of the device as a pathogen mitigator. Alternative architecture and techniques may also be employed.

### PRIORITY CLAIM/CROSS REFERENCE TO RELATED APPLICATION(S)

This Patent Document claims priority under 35 U.S.C. § 119 to U.S. Provisional App. Ser. No. 63/100,687, filed Mar. 26, 2020, and entitled, “Nose Virus Inhibitor”, which is incorporated herein by reference in its entirety.

### BACKGROUND

In recent years, it has been discovered that pathogens, rhinoviruses, in particular, may be particularly prone to survive and replicate in colder air locations of the human body. So, for example, while a human body core temperature of 98.6° F. may be somewhat effective in mitigating pathogen survival and replication, colder air in nostrils, maybe closer to 78° F., and less effective in mitigation.

With this information in mind, the concept of hyperthermic treatment as applied to the nostrils has been proposed. For example, it is believed that holding nostril temperatures above 100° F. for about 20 minutes may be sufficient to reduce pathogen reproduction by 90% or more. Thus, the pathogen would eventually die. Of course, this would not only be for the benefit of the infected person but would also reduce the likelihood of pathogen spread to others.

Unfortunately, the theory of introducing hyperthermia to a nose or nostrils may not be practical through conventional means, particularly for extended period of time. For example, consider a conventional heating patch that might be used to apply hyperthermia to a person at a skin location adjacent sore muscles. These types of patches generally utilize powdered iron and/or other suitable substances to generate heat upon exposure to surrounding air. Powdered iron in particular, may oxidizes fairly rapidly upon exposure to air and oxygen. In this way, heat in excess of about 130° F. may be generated.

Generating heat to this degree may be suitable for a spread out area across a shoulder, back or other muscular location. However, at the discrete location of a user's nose, this level of heat may prove to be a significant irritant. Further, the discreteness in combination with a lack of adjacent depth of muscular tissue to absorb the heat, the skin of the nose may undergo a degree of burning. As a result, it is unlikely that the user would leave such a wearable device in place for a sufficient period of time to allow the device to serve as a practical pathogen mitigator. More specifically, it is unlikely that the user would leave such a device in place for the substantial duration of a day.

In addition to the disadvantage of discomfort, a conventional heat patch may still lack the ability to serve as an effective sterilizing device. That is, even for a user wearing such a device throughout the substantial duration of a day, there remains the likelihood that such a device would be ineffective in terms of mitigating pathogen replication. This is because such devices generally rapidly gain and lose heat, relatively speaking. That is, while such devices may be advertised as effective for heated muscle treatment over a 8-12 hour period or maybe even longer, the reality is that the devices do not maintain a temperature high over such a period. For example, a heat patch that is tailored to deliver a maximum of 135° F. is likely to reach this temperature relatively quickly followed by a steady decline. In terms of applying heat for hyperthermic treatment of a muscle, this may be of some effectiveness. That is, it may be considered to be of some effectiveness whenever the patch is either delivering or effecting a temperature of 98.6° F. or greater. However, the reality is that much of this period is well below 100° F. Thus, if the concept were to be translated over to application at the nose for increasing nostril temperature to over 100° F., much of the effort would be ineffective.

As a practical matter, the concept of utilizing a wearable patch at a user's nose to increase nostril temperature for mitigating pathogens remains unrealistic via conventional means. Utilizing today's known materials and architecture would render no more than an uncomfortable, potentially hazardous device unlikely to be effective for any measurably effective period of time.

### SUMMARY

A pathogen mitigating device is disclosed. The device includes an adhesive substrate for securing at a nose location of a user. A heat generating substance layer is coupled to the substrate to raise a temperature within a nostril of the nose. Further, a temperature modulating substance layer, between the substrate and heat generating layers, is provided to regulate the temperature to a predetermined range for long-term user comfort.

### BRIEF DESCRIPTION OF THE DRAWINGS

Implementations of various structure and techniques will hereafter be described with reference to the accompanying drawings. It should be understood, however, that these drawings are illustrative and not meant to limit the scope of claimed embodiments.

FIG. 1 is a perspective view of an embodiment of a pathogen mitigating wearable device worn by a user.

FIG. 2A is a top view of the pathogen mitigating wearable device of FIG. 1 during activation.

FIG. 2B is a side cross-sectional view of the pathogen mitigating wearable device of FIG. 2B.

FIG. 3 is a side cross-sectional view of the pathogen mitigating wearable device of FIG. 2B employed on a nose of the user of FIG. 1.

FIG. 4 is an alternate embodiment of a pathogen mitigating wearable device incorporated into a mask for the user of FIG. 1.

FIG. 5 is a flow-chart summarizing an embodiment of employing a pathogen mitigating wearable device.

### DETAILED DESCRIPTION

In the following description, numerous details are set forth to provide an understanding of the present disclosure. However, it will be understood by those skilled in the art that the embodiments described may be practiced without these particular details. Further, numerous variations or modifications may be employed, which remain contemplated by the embodiments as specifically described.

Embodiments are described with reference to particular pathogen mitigating wearable devices. In particular, focus is drawn to an adhesively secured nasal strip embodiment. However, a variety of other pathogen mitigating wearable devices may be utilized which take advantage of the architecture and principles detailed herein. For example, masks may benefit from these concepts. So long as a temperature modulating layer is employed between the user and a heat generating layer of the device, appreciable benefit may be realized.

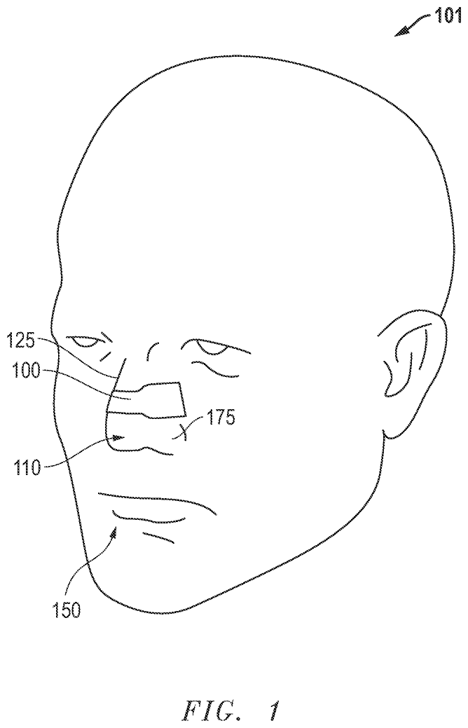

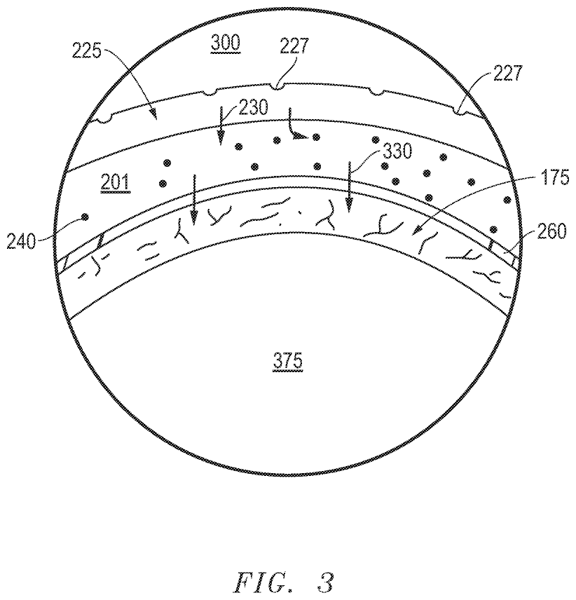

Referring now to FIG. 1, a perspective view of an embodiment of a pathogen mitigating wearable device 100 is illustrated as worn by a user. Specifically, the device 100 is adhesively secured about a bridge 125 of a user's nose 110, reaching each nostril 175. With added reference to FIG. 3, the device 100 may induce an elevated temperature at an interior 375 of the nostril 175 to serve as a pathogen mitigator as detailed further below. However, from the user's perspective, the device 100 may seem much like a conventional nasal dilator or strip upon initial application. That is, apart from, or in addition to pathogen mitigating character, the device 100 may constitute an elongated structure for application of an expanding force for nasal dilation.

As suggested above, the wearable device 100 may be configured to induce an elevated temperature within the nostril 175. Further, as detailed below, the device 100 may do so in a manner that results in a steady application of this hyperthermic treatment, for example, within a steady temperature range for an extended period of time. By way of example only, this may include inducing a nostril interior 375 temperature of between about 98 and 105° F. for a continuous period, in excess of 20 minutes, perhaps 6-8 hours or more (see also FIG. 3). In a sense, the device 100 may be thought of as raising the temperature here from a generally colder range to one more in line with that of the core body temperature (e.g. often considered to be about 98.6° F.).

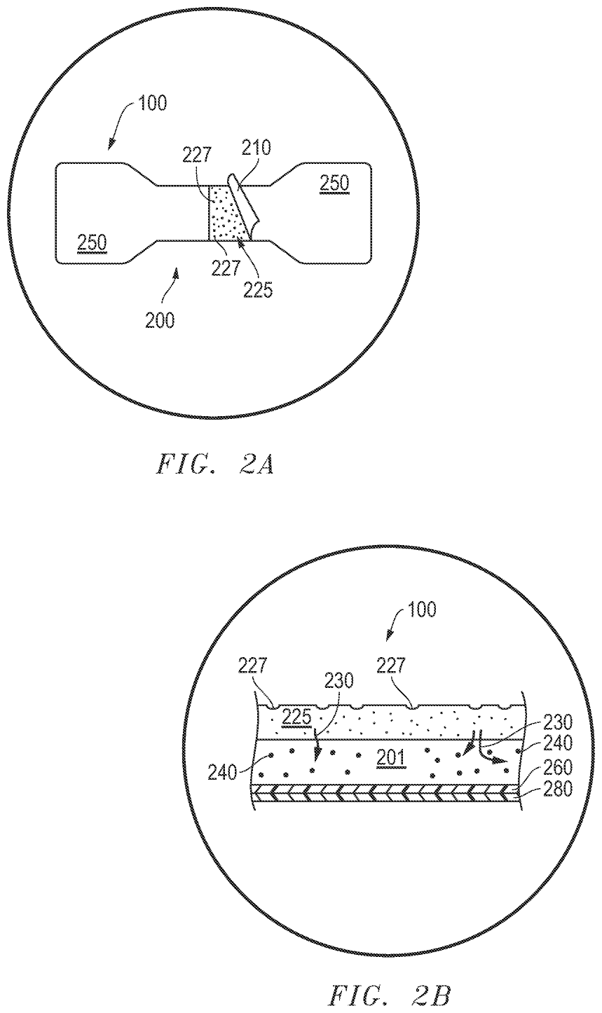

Continuing with reference to FIG. 1, the hyperthermic treatment (HT) is achieved in a modulated manner, wherein a phase change material layer 201 is employed to ensure that temperature at the skin of the nose 125 does not reach an uncomfortably or potentially injuriously high level during the treatment (see also FIG. 2B). Indeed, achieving an elevated temperature at an extremity such as the nose 125 may be done as a matter of warmth and comfort on a cold day, apart from the effectiveness of the device as a pathogen mitigator.

Referring now to FIG. 2A, a top view of the pathogen mitigating wearable device 100 of FIG. 1 is illustrated during activation. For example, in one embodiment, the device 100 is outfitted with a removable isolating strip 210 disposed over an active heat generating layer 225. In the embodiment shown, the active layer 225 includes an iron powder that may undergo oxidation to generate heat upon exposure to oxygen from surrounding air when the isolating strip is removed, generally within a few minutes time. In this manner, the device 100 may remain inactive prior to removal of the strip 210. The strip 210 may be of a paper, wax paper, silicon, polymer, plastic, an oxygen protective foil or other suitable material for isolating the underlying active layer 225 while also being easily removable for a user when the user decides to activate the device 100. In the embodiment shown, the strip 210 includes a slit or separation near the middle of the device 100 to serve as an aid the user in this regard.

The active layer 225 may be heat generating, as noted. As indicated, the exothermic source of heat energy may be supplied by oxidation of powdered iron. However, in other embodiments sodium acetate or other reactive heat generating materials may be utilized. As depicted in the illustration of FIG. 2A, pores 227 may be provided at the surface of the active layer 225. Thus, as the strip 210 is removed, the oxidation of the underlying powdered iron within the layer 225 may be encouraged. Regardless, the generation of exothermic heat for such an embodiment may take place for a duration of 8 hours or longer. Additionally, the layer 225 may also include water, activated charcoal, vermiculite, salt and other secondary materials apart from the active ingredient of the iron powder. Of course, other active ingredients and secondary material combinations may be employed.

In the view of FIG. 2A, the device 100 is of a morphology that includes a narrow bridge portion 200 near the center with wider end regions 250 to the sides. With added reference to FIG. 1, this is in line with the morphology of the user's nose 110 in that the wider end regions 250 provide a greater surface area interface with each nostril 175. Thus, the securely positioned and activated device 100 may be afforded a greater opportunity to effect temperature increase at each nostril interior 375 (see FIG. 3). Further, the overall width of the device 100, end to end, may be between about 0.5 inches and 2.5 inches with a smaller dimension top to bottom as illustrated, again, corresponding to the morphology of the intended placement at the nose 110.

Referring now to FIG. 2B, a side cross-sectional view of the pathogen mitigating wearable device 100 of FIG. 2B is shown. In this view, additional underlying layers 201, 266, 280 are apparent. The temperature modulating layer 201 in particular, plays a role that uniquely facilitates long term functionality and comfort for the user that employs the secured device 100 as illustrated at FIG. 1. Specifically, this layer 201 accommodates a phase change material (PCM) 240. As detailed further below, the PCM 240 may be selected based on a melting point or range that allows it to effectively absorb heat 230 emanating from the adjacent activated layer 225. As also detailed further below, this places a modulator between the heat of this layer 225 and the user's nose 110 as illustrated in FIG. 1. Thus, application of temperature extremes directly to the nose 110 may be avoided. Further, absorbed heat 230 by the PCM 240 and modulating layer 201 may be released over a more extended period of time as melted PCM 240 releases the heat and begins to re-freeze (see FIG. 3).

A variety of biocompatible PCM 240 options are available that may serve the temperature modulating function noted above. For example, paraffin wax, fatty acids and other material choices may be utilized. In one embodiment, an oxidizing powdered iron is utilized as the heat source of the active layer 225, generating about 130° F. The PCM 240 may be a conventional paraffin mixed throughout the modulating layer 201. The PCM 240 may store the initial heat 230 until reaching a melting point of about 103° F. At this time, a lowered heat at about this melting point may begin to enter into the nostril 175 and the interior 375 (see 330 at FIG. 3).

By way of example, in another embodiment, a straight chain saturated hydrocarbon may be utilized for the PCM 240 such as heneicosane. This material may have a melting point of anywhere between about 102° F. to about 109.5° F. Again, materials such as these, with a melting point over 100° F., may be well suited to serve as the PCM 240 to help ensure a raise in temperature within the nostril interior 375 to about 100° F. By the same token, it may also be preferrable to utilize a material with a melting point well below 130° F., perhaps below about 110° F. to ensure long term user comfort. The noted paraffins, saturated hydrocarbons and others may we well suited in this regard.

Continuing with reference to FIG. 2B, an adhesive layer 260 is shown along with protective strip 280. In this case, the strip 280 is merely keeping adhesive of the layer 260 in an isolated state, free of debris or particulate that might affect later adhesive character, when the user is ready to employ the device 100. In one embodiment, the adhesive layer 260 includes a biocompatible, pressure-sensitive material such as elastomeric or acrylic adhesives of a double-sided nature. The protective strip 280 may again be of a suitable paper, wax paper, silicon, polymer or other isolating but readily removable material.

Referring now to FIG. 3, a side cross-sectional view of the pathogen mitigating wearable device 100 of FIG. 2B is illustrated as it is put to use on a nose 110 of the user 101 of FIG. 1. With the isolating strip 210 of FIG. 2A removed, oxygen of the air in the surrounding environment 300 is free to circulate into the heat generating active layer 225. Thus, as described above, an oxidation reaction may ensue to generate substantial heat within the device 100. As the heat dissipates, it may move in the direction of the PCM layer 201 and toward the nostril 175 (see arrows 230). However, upon doing so, this particular heat is filtered through the PCM layer 201 itself. Thus, it may first be absorbed by the actual PCM 240 distributed throughout this layer 201.

By way of example and with reference to the embodiments above, where an iron powder oxidation reaction has generated an initial heat flow 230 of near 130° F., this heat is first absorbed by the PCM 240 throughout the layer 201. In an embodiment where the PCM 240 is paraffin with a melting point of about 103° F., it may be expected that the heat absorbed by the PCM 240 would be sufficient to keep the heat flow 330 toward the nostril 175 at a more comfortable range of below about 103° F. So, for example, consider an embodiment where the heat generating layer 225 is configured to undergo an oxidation reaction emitting a 130° F. heat flow 230 for a period of between about 8 and 12 hours. With added reference to FIG. 1, so long as the paraffin PCM 240 is sufficient in volume to avoid complete melt, temperature within the nostril interior 375 and at the surface of the user's nose 110 will be kept closer to a more comfortable 103° F. by the heat flow 330 from the PCM layer 201. More specifically, given the generally colder nature of the interior 375 in absence of the device 100, the continuous application of 103° F. may lead to a steady interior temperature of more than about 100° F.

As noted above, holding nostril temperatures above 100° F. for about 20 minutes may be sufficient to reduce pathogen reproduction by 90% or more. Thus, the availability of a device 100 that might effectively do this for an 8 to 12 hour period or longer like a conventional heat patch but in a manner that allows practical use at a more delicate extremity such as at a user's nose 110 may be of tremendous benefit. This means that the device 100 is not only effective for pathogen mitigation over the near term but that it may be employed as a practical matter and in a comfortable manner over the long term. That is, the device 100 is not only a pathogen mitigator for a discretely effective time period but for the substantial duration of a given day. This means that the user 101 is not only less prone to contract a pathogen supported illness but that the user 101 is less prone to spread the illness.

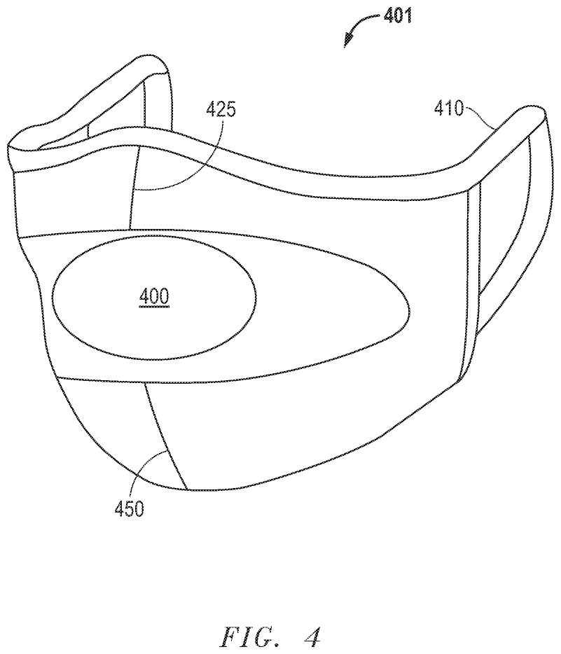

Referring now to FIG. 4, with added reference to FIG. 1, an alternate embodiment of a pathogen mitigating wearable device 400 incorporated into a mask 401 for the user 101. The overall architecture of the mask 401 is conventional in the sense that it covers over both the nose 110 and mouth 150 of the user 101 by way of straps 410 about a user's ears. Therefore, even without the added mitigating device 400, it may still serve as a pathogen mitigator with a front 450 covering over the user's mouth 150 in addition to the fabric region in front of the user's nose 110 (e.g. where the device 400 is located).

In the embodiment shown, the bridge 425 of the mask 401 over the user's nose bridge 125 may be a conventionally suitable mask fabric with the device 400 incorporated near a nostril exit or opening location. The device 400 may be incorporated into the fabric of the mask 401 at this location or provided as a pocket insert. Regardless of this alternate positioning, the device 400 may share the attributes of the pathogen mitigating device 100 of the other embodiments described hereabove. However, additional advantages may also be realized due to the device 400 positioning illustrated in the embodiment of FIG. 4. For example, the device 400 is suspended or held at a location removed from direct contact with the user's nose 110. As a result, the degree or precision of PCM utilized may be less of concern. That is, while it still may be advantageous to utilize a PCM to minimize and/or store heat directed toward vicinity of the nose 110, concern over, for example, 130° F. applied directly to the surface of the nose 110 is eliminated. Indeed, in one embodiment, the use of a PCM layer for the device 400 is avoided altogether.

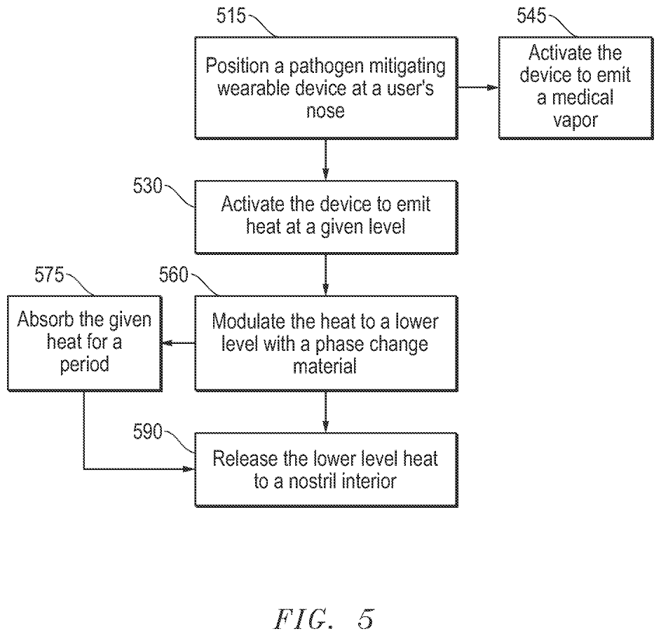

Referring now to FIG. 5, a flow-chart summarizing an embodiment of employing a pathogen mitigating wearable device is depicted. As indicated at 515, the device may be positioned in the vicinity of a user's nose and then activated. As described above, this activation may be to initiate heat emission from the device (see 530). However, in another embodiment, activation may include the emission of a medical vapor as indicated at 545. For example, in one embodiment, anti-viral agent may be released from the device during activation. Citric acid and/or laurel sulfate, particularly when aided by heat induction may serve as suitable vapors in this regard. However, any number of anesthetics, antibiotics, antihistamines and other agents may be employed.

Continuing with reference to FIG. 5, the initial heat activation may be modulated with a phase change material as indicated at 560. Thus, a lower, more comfortable level of pathogen mitigating heat may reach the nose and nostril interior (see 590). Once more, the phase change material may store the initially release heat from the active layer of the device as indicated at 575 before releasing as indicated during phase change material melt. Thus, the delivery and treatment may not only be modulated to a steady temperature but the duration of the treatment may be extended for a substantial period.

Embodiments described hereinabove include devices that allow for the use of a wearable patch-type of device at a user's nose to induce heat. At the same time, the embodiments are uniquely configured to not only mitigate pathogen reproduction by inducing nostril heat but to also mitigate extreme heat. So, for example, the device may be worn long term without undue discomfort. Therefore, a practical manner of heat induced pathogen mitigation may be employed by a user during long term, even daily wear.

The preceding description has been presented with reference to presently preferred embodiments. Persons skilled in the art and technology to which these embodiments pertain will appreciate that alterations and changes in the described structures and methods of operation may be practiced without meaningfully departing from the principle, and scope of these embodiments. For example, additional features may be incorporated into device embodiments described above. These may include the use of outer air and/or oxygen protective packaging, adding an aromatic emission device, a visible temperature indicator, a powered heat source for the heat generating active layer or a variety of other features. Furthermore, the foregoing description should not be read as pertaining only to the precise structures described and shown in the accompanying drawings, but rather should be read as consistent with and as support for the following claims, which are to have their fullest and fairest scope.

I claim:

1. A pathogen mitigating wearable device comprising:
an adhesive substrate for securing at a nose location of a user;
a heat-generating substance layer coupled to the substrate for raising a temperature within a nostril of the nose; and
a temperature modulating substance layer between the substrate and heat generating layers for regulating the temperature to a predetermined range for long-term user comfort.

2. The pathogen mitigating wearable device of claim 1, wherein the predetermined range is an elevated temperature of between about 98° F. and about 105° F.

3. The pathogen mitigating wearable device of claim 1 wherein the heat-generating substance layer accommodates one of iron powder sodium acetate and a powered heat source for the raising of the temperature.

4. The pathogen mitigating wearable device of claim 1, further comprising a removable isolating strip over the heat-generating substance layer to substantially prohibit the raising of the temperature in advance of removal thereof.

5. The pathogen mitigating wearable device of claim 4 with pores at a surface thereof to promote the raising of the temperature upon removal of the isolating strip.

6. The pathogen mitigating wearable device of claim 1 wherein the temperature modulating substance layer accommodates a phase change material for the regulating of the temperature.

7. The pathogen mitigating wearable device of claim 6 wherein the phase change material is selected from a group consisting of paraffin, a fatty acid, a saturated hydrocarbon, heneicosane,

8. The pathogen mitigating wearable device of claim 6 wherein the phase change material has a melting point of over about 100° F.

9. The pathogen mitigating wearable device of claim 8 wherein the phase change material has a melting point of below about 110° F.

10. A pathogen mitigating wearable device comprising:
a heat-generating substance layer for raising a temperature within a nostril of a nose; and
architecture for positioning the heat-generating substance layer adjacent the nose in a manner avoiding direct physical contact therewith.

11. The pathogen mitigating wearable device of claim 10 further comprising one of a visible temperature indicator and an aromatic emission device.

12. The pathogen mitigating wearable device of claim 10 wherein the architecture comprises one of a mask structure for locating the heat-generating substance layer adjacent an opening of the nostril and a temperature modulating substance layer located between the heat-generating substance layer and the nose.

13. The pathogen mitigating wearable device of claim 12 wherein the temperature modulating substance layer accommodates a phase change material for regulating the temperature.

14. The pathogen mitigating wearable device of claim 13 wherein the phase change material is selected from a group consisting of paraffin, a fatty acid, a saturated hydrocarbon and heneicosane.

15. The pathogen mitigating wearable device of claim 13 wherein the phase change material has a melting point of over about 100° F.

16. The pathogen mitigating wearable device of claim 15 wherein the phase change material has a melting point of below about 110° F.

17. A method of pathogen mitigation comprising:
placing a pathogen mitigation device at a nose of a user with a nostril having an initial interior temperature;
activating the device to emit heat at a given temperature;
modulating the heat from the device with a phase change material to reduce the given temperature and facilitate increasing of the interior temperature.

18. The method of claim 17 further comprising absorbing the heat with the phase change material to extend a period of the increasing of the interior temperature.

19. The method of claim 17 further comprising activating the device to emit a medical vapor.

20. The method of claim 17 wherein the increasing of the interior temperature is to a temperature between about 98° F. and about 105° F.
